(19) United States
(12) Patent Application Publication
(10) Pub. No.: US 2007/0076896 A1
(43) Pub. Date: Apr. 5, 2007

(54) Active noise-reduction control apparatus and method

(75) Inventors: Rika Hosaka, Yokohama-shi (JP); Akihiko Enamito, Kawasaki-shi (JP); Kenji Kojima, Chiba-shi (JP); Shinya Kijimoto, Fukuoka-shi (JP)

Correspondence Address: Charles N.J. Ruggiero;Ohlandt, Greeley, Ruggiero & Perle, L.L.P., 10th Floor, One Landmark Square, Stamford, CT, 06901-2682, US

(73) Assignee: KABUSHIKI KAISHA TOSHIBA

(21) Appl. No.: 11/501,332

(22) Filed: Aug. 7, 2006

(30) Foreign Application Priority Data
Sep. 28, 2005 (JP) 2005-282804

(51) Int. Cl. A61F 11/06; G10K 11/16; H03B 29/00

(52) U.S. Cl. 381/71.11; 381/71.1

(57) ABSTRACT

Apparatus includes generator generating reference signal based on noise emitted from sound source, detector detecting level of reference signal and change in level, unit comparing change with threshold-value range and produce compared result, filter filtering reference signal, adaptive filter having variable filter coefficient, unit updating filter coefficient according to change of level of reference signal for obtaining an updated filter coefficient, unit stopping updating of filter coefficient in response to compared result when change falls outside threshold-value range, unit storing updated filter coefficient each time filter coefficient is updated, generator generating control signal using stored filter coefficient, unit generating control sound based on control signal, microphone detecting synthesis sound pressure of control sound and noise to produce an error signal, and unit setting stored filter coefficient to more accurate coefficient than stored filter coefficient based on error signal, and signal acquired by filtering control signal through filter.

### CROSS-REFERENCE TO RELATED APPLICATIONS

This application is based upon and claims the benefit of priority from prior Japanese Patent Application No. 2005-282804, filed Sep. 28, 2005, the entire contents of which are incorporated herein by reference.

### BACKGROUND OF THE INVENTION

1. Field of the Invention

The present invention relates to active noise reduction for reducing, using reference signal supply means, an error microphone and control speaker, the level of a noise source, such as an unsteady sound having a varying sound pressure level, an intermittent sound including silent portions and emitted by a sound source that intermittently stops, or a sound emitted from a moving sound source. More particularly, it relates to a control method and apparatus for suppressing sound pressure at the position of an error microphone.

2. Description of the Related Art

In active control, when a Filtered-X least mean square (LMS) algorithm as a generally used arithmetic algorithm is used, noise that greatly fluctuates in sound pressure level, or error factors in the varying acoustic path of a moving sound source may lead to degradation of control effect, whereby the control is inevitably destabilized. Further, since the LMS algorithm is of a gradient-method type, it does not require a large number of computations and hence is very stable. However, it has a fatal problem that a lot of time is required for amplitude convergence. Because of this problem, the LMS algorithm is hard to apply to level-varying noise or moving noise.

In light of the above, direct algorithms have been contrived. For example, in an algorithm directed to a steady sound emitted from a moving sound source (disclosed in, for example, Kijimoto et al., “Active Audio Control Utilizing Algorithm That Follows Change in Error Route at High Speed,” published by the Japan Society of Mechanical Engineers, 14^{th} Environment Engineering General Symposium 2004, Lecture Articles, pp. 42-45; Sasaki et al., “Active Audio Control of Noise From the Outside,” published by the Japan Society of Mechanical Engineers, 13^{th} Environment Engineering General Symposium 2003, Lecture Articles, pp. 49-52), a single fixed filter K and its adaptive filters K and D are installed as well as an adaptive filter C for updating the coefficient of a control filter C, and a virtual error signal is generated based on an error microphone signal to update the coefficients of the adaptive filters. Since the same gradient-type LMS algorithm as the conventional Filtered-X algorithm is used for updating the coefficients, the convergence speed cannot be improved, either. However, there is no error route as a control-destabilizing factor, and hence no amplitude divergence occurs even in the case of a sound emitted form a moving sound source, which means that stable control can be realized.

The method developed to aim higher-speed amplitude convergence in the above stable control state is a direct fast transversal filter (FTF) method.

However, even the direct FTF method exhibits instability when it is used to process an unsteady sound that significantly varies in level, therefore cannot easily follow it.

### BRIEF SUMMARY OF THE INVENTION

In accordance with a first aspect of the invention, there is provided an active noise-reduction control apparatus comprising: a reference signal generator which generates a reference signal based on noise emitted from a sound source; a detector which detects a level of the reference signal and a change in the level; a comparison unit configured to compare the change with a threshold-value range and produce a compared result; an adaptive filter configured to filter the reference signal, the adaptive filter having a variable filter coefficient; an updating unit configured to update the filter coefficient according to the change of the level of the reference signal for obtaining an updated filter coefficient; a stop unit configured to stop updating of the filter coefficient in response to the compared result when the change falls outside the threshold-value range; a storage unit configured to store the updated filter coefficient each time the filter coefficient is updated; a control signal generator which generates a control signal using the stored filter coefficient; a control-sound source unit configured to generate a control sound based on the control signal; an error microphone which detects synthesis sound pressure of the control sound and the noise to produce an error signal; and a setting unit configured to set the stored filter coefficient to a more accurate coefficient than the stored filter coefficient based on the error signal, and a signal acquired by filtering the control signal through the adaptive filter.

In accordance with a second aspect of the invention, there is provided an active noise-reduction control apparatus comprising: a reference signal generator which generates a reference signal based on noise emitted from a sound source; a detector which detects a level of the reference signal and a change in the level; a comparison unit configured to compare the change with a threshold-value range and produce a compared result; an adaptive filter configured to filter the reference signal, the adaptive filter having a variable filter coefficient; an initialization unit configured to initialize the filter coefficient in response to the compared result when the change falls outside the threshold-value range; a control signal generator which generates a control signal using the filter coefficient; a control-sound source unit configured to generate a control sound based on the control signal; an error microphone which detects synthesis sound pressure of the control sound and the noise to produce an error signal; and a setting unit configured to set the filter coefficient to a more accurate coefficient than the filter coefficient based on the error signal, and a signal acquired by filtering the control signal through the adaptive filter.

In accordance with a third aspect of the invention, there is provided an active noise-reduction control apparatus comprising: a reference signal generator which generates a reference signal based on noise emitted from a sound source; a control-sound source unit configured to generate a control sound used for controlling reduction of the noise; an error microphone which detects synthesis sound pressure of the control sound and the noise to produce an error signal; a computation unit configured to compute an estimated error based on the reference signal and the error signal; an adaptive filter configured to filter the reference signal, the adaptive filter having a variable filter coefficient; an adjustment unit configured to adjust the filter coefficient based on the estimated error; a control signal generator which generates a control signal using the filter coefficient; and a setting unit configured to set the filter coefficient to a more accurate coefficient than the filter coefficient based on the error signal, and a signal acquired by filtering the control signal through the adaptive filter, the control-sound source unit generating the control sound based on the control signal.

In accordance with a fourth aspect of the invention, there is provided an active noise-reduction control apparatus comprising: a reference signal generator which generates a reference signal based on noise emitted from a sound source; a detector which detects a level of the reference signal and a change in the level; a comparison unit configured to compare the change with a threshold-value range and produce a compared result; a first adaptive filter configured to filter the reference signal, the first adaptive filter having a first variable filter coefficient; a first updating unit configured to update the first variable filter coefficient according to the change of the level of the reference signal for obtaining an updated first variable filter coefficient; a first stop unit configured to stop updating of the first variable filter coefficient in response to the compared result when the change falls outside the threshold-value range; a first control signal generator which generates a first control signal using the first variable filter coefficient; a first control-sound source unit configured to generate a first control sound based on the first control signal; a first error microphone which detects first synthesis sound pressure of the first control sound and the noise to produce a first error signal; a first setting unit configured to set the first variable filter coefficient to a more accurate coefficient than the first variable filter coefficient based on the first error signal, and a signal acquired by filtering the first control signal through the first adaptive filter; a second adaptive filter configured to filter the reference signal, the second adaptive filter having a second variable filter coefficient; a second updating unit configured to update the second variable filter coefficient according to the change of the level of the reference signal for obtaining an updated second variable filter coefficient; a second stop unit configured to stop updating of the second variable filter coefficient in response to the compared result when the change falls outside the threshold-value range; a second control signal generator which generates a second control signal using the second variable filter coefficient; a second control-sound source unit configured to generate a second control sound based on the second control signal; a second error microphone which detects second synthesis sound pressure of the second control sound and the noise to produce a second error signal; a second setting unit configured to set the second variable filter coefficient to a more accurate coefficient than the second variable filter coefficient based on the second error signal, and a signal acquired by filtering the second control signal through the second adaptive filter.

In accordance with a fifth aspect of the invention, there is provided an active noise-reduction control method comprising: generating a reference signal based on noise emitted from a sound source; detecting a level of the reference signal and a change in the level; comparing the change with a threshold-value range and producing a compared result; preparing an adaptive filter configured to filter the reference signal, the adaptive filter having a variable filter coefficient; updating the filter coefficient according to the change of the level of the reference signal for obtaining an updated filter coefficient; stopping updating of the filter coefficient in response to the compared result when the change falls outside the certain threshold-value range; preparing a storage unit configured to store the updated filter coefficient each time the filter coefficient is updated; generating a control signal using the stored filter coefficient; generating a control sound based on the control signal; detecting synthesis sound pressure of the control sound and the noise to produce an error signal; and setting the stored filter coefficient to a more accurate coefficient than the stored filter coefficient based on the error signal, and a signal acquired by filtering the control signal through the adaptive filter.

In accordance with a sixth aspect of the invention, there is provided an active noise-reduction control method comprising: generating a reference signal based on noise emitted from a sound source; detecting a level of the reference signal and a change in the level; comparing the change with a threshold-value range and producing a compared result; preparing an adaptive filter configured to filter the reference signal, the adaptive filter having a variable filter coefficient; initializing the filter coefficient in response to the compared result when the change falls outside the threshold-value range; generating a control signal using the filter coefficient; generating a control sound based on the control signal; detecting synthesis sound pressure of the control sound and the noise to produce an error signal; and setting the filter coefficient to a more accurate coefficient than the filter coefficient based on the error signal, and a signal acquired by filtering the control signal through the adaptive filter.

In accordance with a seventh aspect of the invention, there is provided an active noise-reduction control method comprising: generating a reference signal based on noise emitted from a sound source; generating a control sound used for controlling reduction of the noise; detecting synthesis sound pressure of the control sound and the noise and producing an error signal; computing an estimated error based on the reference signal and the error signal; preparing an adaptive filter configured to filter the reference signal, the adaptive filter having a variable filter coefficient; adjusting the filter coefficient based on the estimated error; generating a control signal using the filter coefficient; and setting the filter coefficient to a more accurate coefficient than the filter coefficient based on the error signal, and a signal acquired by filtering the control signal through the adaptive filter, generating the control sound based on the control signal.

In accordance with an eighth aspect of the invention, there is provided an active noise-reduction control method comprising: generating a reference signal based on the noise emitted from a sound source; detecting a level of the reference signal and a change in the level; comparing the change with a threshold-value range and producing a compared result; preparing a first adaptive filter configured to filter the reference signal, the first adaptive filter having a first variable filter coefficient; updating the first variable filter coefficient according to the change of the level of the reference signal for obtaining an updated first variable filter coefficient; stopping updating of the first variable filter coefficient in response to the compared result when the change falls outside the threshold-value range; generating a first control signal using the first variable filter coefficient; generating a first control sound based on the first control signal; detecting first synthesis sound pressure of the first control sound and the noise and producing a first error signal; setting the first variable filter coefficient to a more accurate coefficient than the first variable filter coefficient based on the first error signal, and a signal acquired by filtering the first control signal through the first adaptive filter; preparing a second adaptive filter configured to filter the reference signal, the second adaptive filter having a second variable filter coefficient; updating the second variable filter coefficient according to the change of the level of the reference signal for obtaining an updated second variable filter coefficient; stopping updating of the second variable filter coefficient in response to the compared result when the change falls outside the threshold-value range; generating a second control signal using the second variable filter coefficient; generating a second control sound based on the second control signal; detecting second synthesis sound pressure of the second control sound and the noise to produce a second error signal; setting the second variable filter coefficient to a more accurate coefficient than the second variable filter coefficient based on the second error signal, and a signal acquired by filtering the second control signal through the second adaptive filter.

### BRIEF DESCRIPTION OF THE SEVERAL VIEWS OF THE DRAWING

The file of this patent contains at least one drawing executed in color. Copies of this patent with color drawings will be provided by the Patent and Trademark Office upon request and payment of the necessary fee.

FIG. 1A is a block diagram illustrating an active noise-reduction control apparatus according to a first embodiment of the invention;

FIG. 1B is a block diagram illustrating a general adaptive filter unit;

FIG. 2 is a block diagram illustrating a first example of the active noise-reduction control apparatus FIG. 1A;

FIG. 3A is a view illustrating a typical unsteady sound, e.g., the sound of a snore, as a level reduction target;

FIG. 3B is a view illustrating the effect acquired when the active noise-reduction control apparatus of FIG. 2 is used;

FIG. 4 is a control block diagram corresponding to the active noise-reduction control apparatus of FIG. 2;

FIG. 5A is a graph illustrating simulation results that are acquired using an LMS method when an adaptation operation is temporarily stopped at a low noise level portion;

FIG. 5B is a graph illustrating simulation results that are acquired using the LMS method when the adaptation operation is continued;

FIG. 6 is a block diagram illustrating a second example of the active noise-reduction control apparatus FIG. 1A;

FIG. 7 is a block diagram illustrating an active noise-reduction control apparatus according to a second embodiment of the invention;

FIG. 8 is a block diagram illustrating a first example of the active noise-reduction control apparatus FIG. 7;

FIG. 9 is a block diagram illustrating a second example of the active noise-reduction control apparatus FIG. 7;

FIG. 10 is a view illustrating a system for performing experiments using the active noise-reduction control apparatus FIG. 9;

FIG. 11 is a graph illustrating a time-series waveform acquired when a direct LMS method is utilized;

FIG. 12 is a graph illustrating a control effect in units of frequencies acquired when the direct LMS method is utilized;

FIG. 13 is a graph illustrating a time-series waveform acquired when a direct FTF method is utilized;

FIG. 14 is a graph illustrating a control effect in units of frequencies acquired when the direct FTF method is utilized;

FIG. 15 is a view illustrating the degrees of the effect of reducing, for example, noise of snoring, acquired when the active noise-reduction control apparatus is in the off state, when the LMS method is utilized, and when the FTF method is utilized;

FIG. 16 is a view illustrating the degrees of the error microphone effect of reducing the level of a reduction target, acquired when the active noise-reduction control apparatus is in the off state, when the LMS method is utilized, and when the FTF method is utilized;

FIG. 17 is a block diagram illustrating an active noise-reduction control apparatus according to a third embodiment of the invention;

FIG. 18 is a block diagram illustrating a first example of the active noise-reduction control apparatus FIG. 17;

FIG. 19 is a block diagram illustrating a second example of the active noise-reduction control apparatus FIG. 17;

FIG. 20A is a graph illustrating the control effect acquired when coefficient λ is updated in accordance with the number of times of control sampling implemented by the active noise-reduction control apparatus FIG. 19;

FIG. 20B is a graph illustrating a history of changes in coefficient λ acquired in accordance with the number of times of control sampling;

FIG. 21 is a block diagram illustrating an active noise-reduction control apparatus according to a fourth embodiment of the invention;

FIGS. 22A and 22B are views useful in explaining the stroke assumed when two error microphones are installed as shown in FIG. 21;

FIG. 23 is a view illustrating a system structure for performing experiments for verifying the advantage of the apparatus of FIG. 21;

FIG. 24A is a graph illustrating the effect of reducing white noise by a first error microphone;

FIG. 24B is a graph illustrating the effect of reducing white noise by a second error microphone;

FIG. 25A is a graph illustrating the effect of reducing the noise of snoring by the first error microphone;

FIG. 25B is a graph illustrating the effect of reducing the noise of snoring by the second error microphone; and

FIG. 26 is a view illustrating the effect of reducing the noise of snoring by the first and second error microphones in units of frequency bands.

### DETAILED DESCRIPTION OF THE INVENTION

Active noise-reduction control apparatuses and methods according to embodiments of the invention will be described in detail with reference to the accompanying drawings. Firstly, an LMS algorithm (LMS method) and direct algorithm will be described briefly.

Filtered-X LMS algorithm is a generally used arithmetic algorithm in active control. In this algorithm, the characteristic G (error route) of the spatial transmission route between a control-sound source and an error microphone is beforehand identified, and control filter C is updated based on the assumption that the characteristic is known and time-invariant. Namely, G is set as a fixed filter coefficient.

In contrast, the direct algorithm in which the characteristic G of the spatial transmission route between the control-sound source and error microphone is not beforehand identified, a plurality of control filters and adaptive filters are used to update control filter C instead of the error route G.

The active noise-reduction control apparatuses and methods can perform control for suppressing error microphone sound pressure in a stable manner at high speed, without diverging the sound pressure.

### First Embodiment

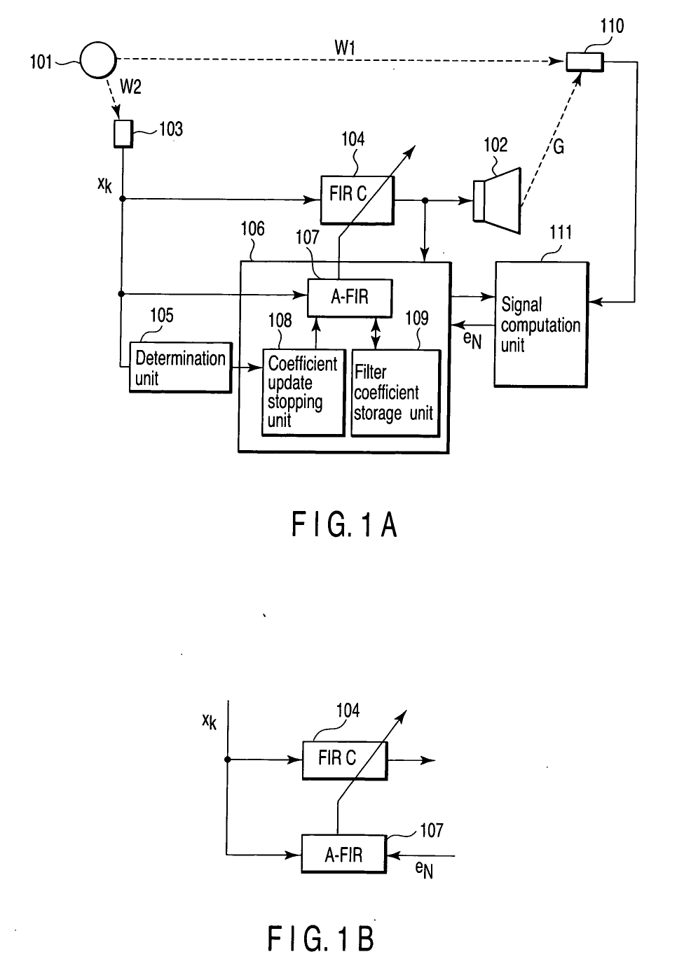

Referring to FIG. 1A, an active noise-reduction control apparatus according to a first embodiment of the invention will be described.

The active noise-reduction control apparatus of the first embodiment comprises a control-sound source unit 102, reference signal generator 103, digital filter arithmetic unit 104, determination unit 105, filter coefficient updating unit 106, error microphone 110 and signal computation unit 111. The filter coefficient updating unit 106 includes an adaptive filter unit 107, coefficient update stopping unit 108 and filter coefficient storage unit 109. The active noise-reduction control apparatus of the first embodiment is used to reduce a to-be-reduced noise (target noise) 101 emitted from a sound source.

The reference signal generator 103 receives the target noise 101, generates a reference signal based on the target noise 101, and supplies the reference signal to the digital filter arithmetic unit 104, determination unit 105 and filter coefficient updating unit 106.

The determination unit 105 detects the level (absolute voltage) of a reference signal, and a level change (relative voltage) indicating the degree to which the level of the reference signal is changed with lapse of time. Specifically, the determination unit 105 sets a certain threshold-value range, and compares a change in the level of the reference signal with the threshold-value range, and outputs, to the coefficient update stopping unit 108, a signal indicating whether the level change falls within the threshold-value range.

The filter coefficient updating unit 106 updates the coefficient of the digital filter arithmetic unit 104, based on the reference signal.

Upon receiving the determination result of the determination unit 105, the coefficient update stopping unit 108 stops update of the coefficient of the adaptive filter unit 107 in accordance with the determination result. For instance, upon receiving, from the determination unit 105, a signal indicating that the level change falls outside a preset threshold-value range, the coefficient update stopping unit 108 stops update of the coefficient of the adaptive filter unit 107. A more specific example will be described later with reference to equation (Eq. 1).

The filter coefficient storage unit 109 stores the coefficient of the adaptive filter unit 107 whenever the coefficient is updated. Accordingly, the storage unit 109 also stores the coefficient of the adaptive filter unit 107 acquired immediately before the coefficient update stopping unit 108 stops the update of the filter coefficient.

The adaptive filter unit 107 updates the filter coefficient based on the signal output from the signal computation unit 111, and outputs the updated filter coefficient to the digital filter arithmetic unit 104.

The control-sound source unit 102 generates a control sound for reducing the to-be-reduced noise 101.

The error microphone 110 detects the synthesis sound pressure of the control sound from the control-sound source unit 102 and the to-be-reduced noise 101.

The digital filter arithmetic unit 104 receives the filter coefficient updated by the adaptive filter unit 107, and performs filtering processing on the reference signal based on the received coefficient, thereby generating a control signal used by the control-sound source unit 102 to generate a control sound.

The signal computation unit 111 computes a signal for outputting a signal necessary to change the filter coefficient, based on a signal from the filter coefficient updating unit 106 and an error signal from the error microphone 110. The output signal of the filter coefficient updating unit 106 is acquired by, for example, filtering a control signal through the adaptive filter unit 107.

The operation of the coefficient update stopping unit 108 will now be described.

Upon receiving, from the determination unit 105, a signal indicating the stop of the coefficient update, the coefficient update stopping unit 108 stops transfer, to the digital filter arithmetic unit 104, of the coefficient updated by the adaptive filter unit 107.

A specific example will be described, referring to the block diagram of FIG. 1B that shows a general adaptive filter unit. When stopping the coefficient update, the coefficient update stopping unit 108 sets, to 0, constant μ included in the following equation (Eq. 1) as an adaptive-filter update expression. At this time, the adaptive filter unit 107 does not stop update computation and transfer of the update result to the digital filter arithmetic unit 104. However, the difference between before and after the transfer is zero, which is equivalent to the stop of the coefficient update.
<C>_{N+1}=<C>_{N}−μ·e_{N}·<x>ₖ (Eq. 1)
where <A> is vector A, and subscript N of filter C is the number of times of update. Assuming that the number of times of update at present is N, the left-hand side indicates updated (future) filter C. Update of the k^{th} component (scalar value) of filter C is expressed as follows:
C(k)_{N+1}=C(k)_{N}−μ·e_{N}(n)·x(n−k+1), (k=1, 2, . . . , M), (C₁, C₂, . . . , C_{M})^{T}_{N+1}=(C₁, C₂, . . . , C_{M})^{T}_{N}−μ·e_{N}·(x(n), x(n−1), . . . , x(n−M+1))^{T}

Concerning filters K and L described later, the subscripts indicate the same meaning as the above.

In the coefficient update stopped state, no influence of an external input x(n) is exerted, therefore stable control is possible. When the change in the level of the reference signal is returned from a high-level range to the threshold-value range, the adaptive filter coefficient stored in the storage unit immediately before the coefficient update stopping is started is read therefrom, thereby resuming the filter coefficient update, or returning constant μ to the original value and resuming the filter coefficient update.

As described above, in the first embodiment, suppression of noise pressure in the error microphone 110 is realized without identifying the error route (spatial transmission function) between the error microphone 110 and to-be-reduced noise 101. Concerning the noise falling outside the threshold-value range set in the determination unit 105, suppression of noise pressure in the error microphone 110 is realized by stopping the coefficient changing operation of the filter coefficient updating unit 106, even if the to-be-reduced noise 101 is an unsteady sound of a greatly varying level, intermittent sound (including silent portions) emitted from a sound source that intermittently stops, or sound emitted from a moving sound source.

### FIRST EXAMPLE

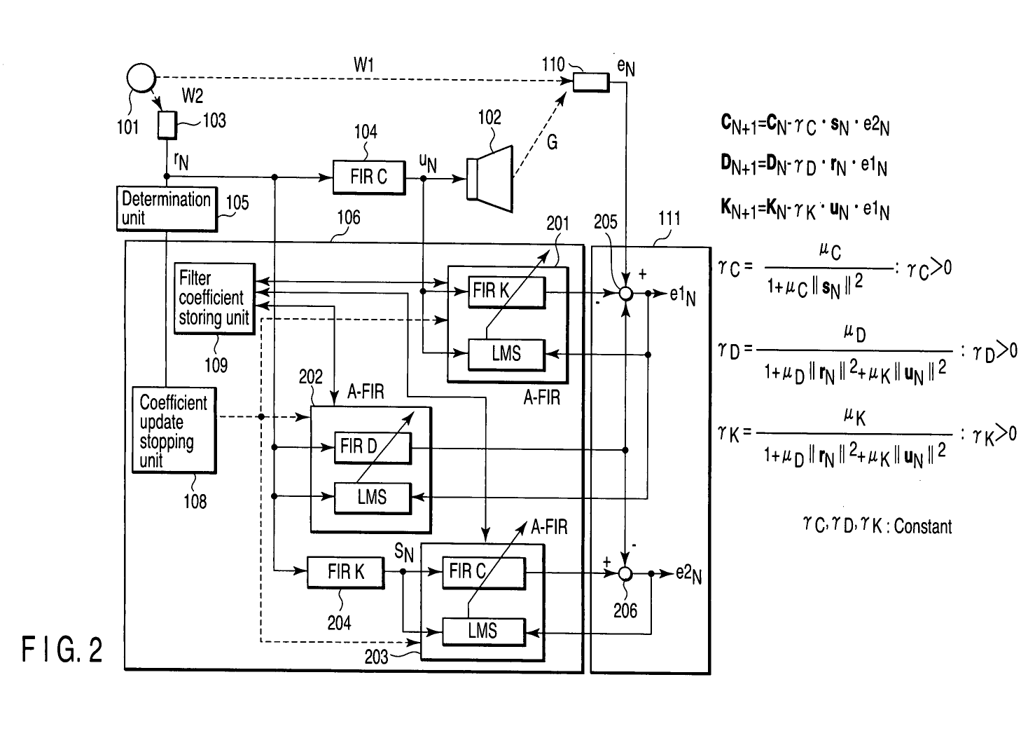

Referring to FIG. 2, a description will be given of a first example of the active noise-reduction control apparatus according to the first embodiment. In the first example, elements similar to those described above are denoted by corresponding reference numbers, and are not described.

In the first example of the active noise-reduction control apparatus, the filter coefficient updating unit 106 includes, as the adaptive filter unit 107, adaptive filter units 201, 202 and 203 and fixed filter arithmetic unit 204. The adaptive filter unit 201 includes a control filter K and LMS computation unit, the adaptive filter unit 202 includes a control filter D and LMS computation unit, and the adaptive filter unit 203 includes a control filter C and LMS computation unit. Based on the outputs of the three adaptive filter units 201 to 203 and the output of the error microphone 110, the signal computation unit 111 two virtual error signals (e1_{N}, e2_{N}) necessary to update the coefficients of the adaptive filter units 201 to 203. The LMS method is applied to coefficient updating computation for the adaptive filter units 201 to 203, thereby realizing suppression of error microphone sound pressure.

Assume here that the sound pressure of the noise source has greatly changed, and the threshold value, i.e., threshold value ξ given by the following equation (Eq. 2), of the determination unit 105 has come to be 0.01 (corresponding to a dynamic range of 10 dB) or less. At this time, the coefficient update stopping unit 108 stops the adaptive coefficient updating operation. However, since it is necessary to continue an adaptive coefficient updating operation until a preset time elapses immediately after the adaptive coefficient updating operation is started, the initial value for ξ is set to, for example, 2.
ξ=κ·ξ+(1−κ)·x²≦0.01 (Eq. 2)

κ=0.999

where x is the amplitude of a reference signal supplied from the reference signal generation unit 103.

This equation means to acquire ξ at the left-hand side, i.e., a new value of ξ, by updating ξ at the right-hand side, i.e., the present value of ξ.

When stopping the coefficient updating operation, the coefficient update stopping unit 108 does not transfer, to the digital filter arithmetic unit 104, coefficient C_{N+1} newly acquired by the adaptive filter unit 203. It is sufficient if the coefficient update stopping unit 108 at least stops the update of the control filter C included in the adaptive filter unit 203. However, simultaneously with the stop, the coefficient update stopping unit 108 may stop the update of the control filters K and D included in the adaptive filter units 201 and 202.

Alternatively, the coefficient update stopping unit 108 may set, to 0, constant γ_{C} included in an adaptive filter update computation expression for the control filter C. It is sufficient if the coefficient update stopping unit 108 at least sets, to 0, the adaptive computation constant for the control filter C. However, simultaneously with this constant, the coefficient update stopping unit 108 may set, to 0, constants γ_{K} and γ_{D} for the control filters K and D.

γ_{C}, γ_{D} and γ_{K} are given by the following equations that include constant μ_{C}, μ_{D} and μ_{K}:
<C>_{N+1}=<C>_{N}−γ_{C}·<s>_{N}·e2_{N}
<D>_{N+1}=<D>_{N}−γ_{D}·<r>_{N}·e1_{N}
<K>_{N+1}=<K>_{N}−γ_{K}·<u>_{N}·e1_{N}
γ_{C}=μ_{C}/(1+μ_{C}∥<s>_{N}∥²);γ_{C}>0
γ_{D}=μ_{D}/(1+μ_{D}∥<r>_{N}∥²+μ_{K}∥<u>_{N}∥²); γ_{D}>0
γ_{K}=μ_{K}/(1+μ_{D}∥<r>_{N}∥²+μ_{K}∥<u>_{N}∥²); γ_{K}>0
The details of the adaptive control computation will be expressed by the following equations (Eq. 3):
<C>_{N}=(C(1), C(2), . . . , C(M))^{T}_{N}, (k=1, 2, . . . , M) (Eq. 3)
<s>_{N}=(s(1), s(2), . . . , s(M))^{T}_{N}, (k=1, 2, . . . , M) (Eq. 3)
where <C>_{N} is M column vectors acquired when the number of update operations is N (the N^{th} operation is the present update operation), and <s>_{N} is similarly M column vectors.

Further, e2_{N} in the following equation is the scalar value (a single data item acquired by analog-to-digital conversion) of the second virtual error signal of the N^{th} (present) update operation.
<C>_{N+1}=<C>_{N}−γ_{C}·<s>_{N}·e2_{N} (Eq. 3)

Accordingly, the above equation can be rewritten into the following equation, assuming that the k^{th} coefficient (scalar value) is C(k):
C(k)_{N+1}=C(k)_{N}−_{C}·s(n−k+1)·e2_{N}(n), (k=1, 2, . . . , M−1) (Eq. 3)

For instance, the first coefficient acquired after the update operation is a value obtained by subtracting, from the present first filter C, a particular value. The particular value is acquired by multiplying, by coefficient γ_{C}, a value that is obtained by multiplying the currently extracted S by the scalar value of e2.
C(1)_{N+1}=C(1)_{N}−γ_{C}·s(n)_{N}·e2_{N}(n) (Eq. 3)

In the same way as the above, the coefficient update stopping unit 108 updates all filter coefficients ranging from k=2 to M−1.

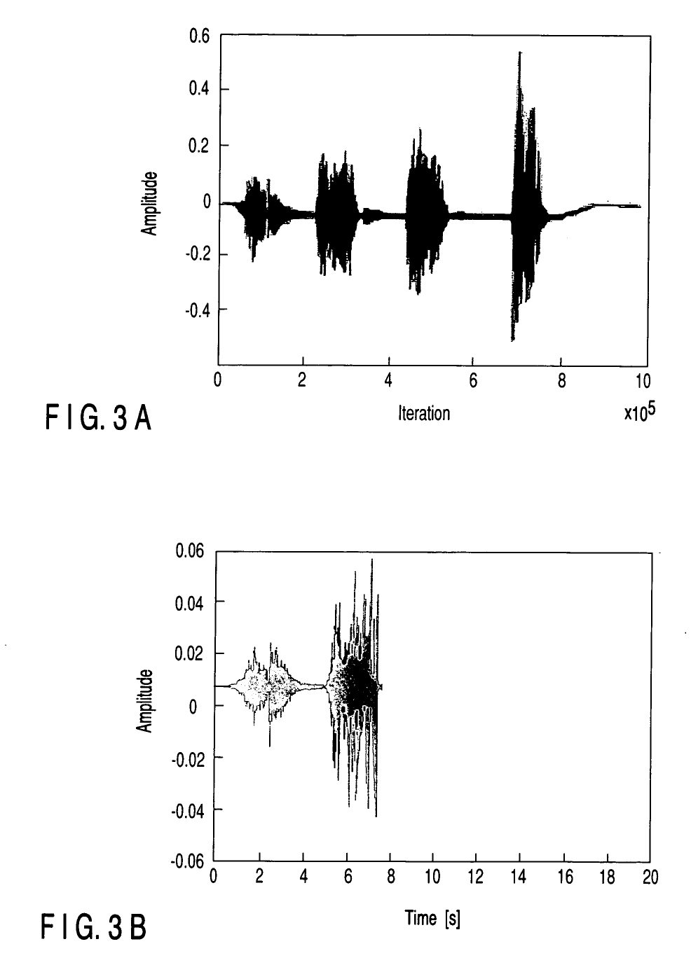

Referring then to FIG. 3B, a description will be given of an example in which the conventional method and the method (algorithm) of the embodiment are applied to the noise of snoring, typical unsteady noise, shown in FIG. 3A, thereby proving the advantage of the algorithm employed in the embodiment.

Firstly, the conventional Filtered-X algorithm will be described. In FIG. 1, the spatial transmission function W1 between the noise source 101 and error microphone 110, the spatial transmission function W2 between the noise source 101 and reference signal generator 103, and the spatial transmission function G between the speaker 102 and error microphone 110 are set to actually measured values. FIG. 3B shows results of simulation made by the conventional method, using pre-recorded noise of snoring as the noise source. The black lines indicate the amplitudes acquired before control, and the gray lines indicate the amplitude acquired after control. As shown in FIG. 3B, in the conventional method, the first noise pattern (first noise pattern of snoring) does not converge as a result of a control delay, and the second noise pattern (second noise pattern of snoring), which shows greater variations than the first noise pattern, diverges.

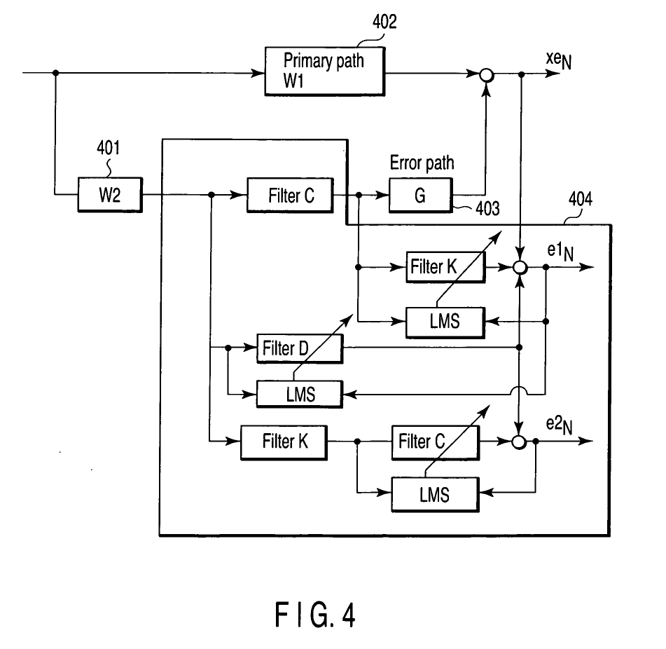

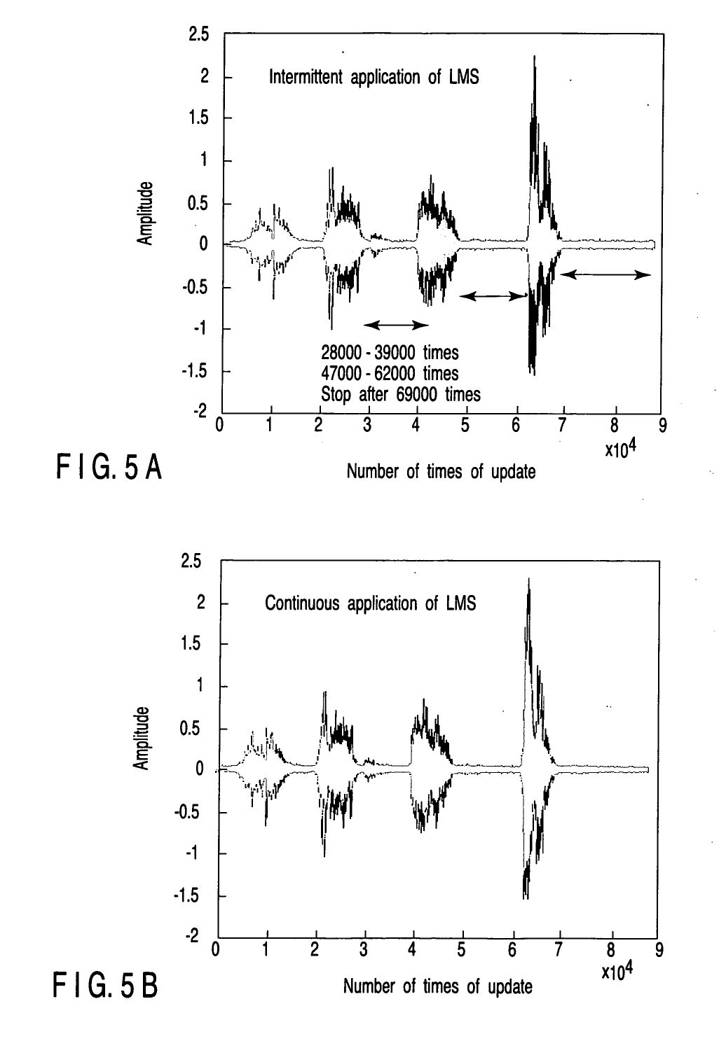

Referring to FIGS. 5A and 5B, a description will be given of the simulation results acquired when the noise-reduction control apparatus of FIG. 2 applies white noise as external noise during emission of no sound, using the direct LMS method, and the validity of the adaptive coefficient update stop operation is estimated using the threshold value of the determination unit. The simulation results are acquired under the control shown in FIG. 4 corresponding to the active noise-reduction control apparatus of FIG. 2, using the values measured in the actual environment. FIG. 4 shows, as an example, an algorithm for a moving unsteady sound.

The control block shown in FIG. 4 is characterized in that a single fixed filter K, its adaptive filter K, an adaptive filter D are installed as well as the adaptive filter C for updating the coefficient of the control filter C, a virtual error signal is generated based on an error microphone signal, and the adaptive filter coefficients are updated. Since an LMS algorithm of the same gradient method as the conventional Filtered-X algorithm is used for the coefficient update computation shown in FIG. 4, the amplitude convergence speed cannot uniformly be improved. However, there is no error route as a control-destabilizing factor, and hence no amplitude divergence occurs even in the case of a sound emitted from a moving sound source, which means that stable control can be realized. Direct FTF methods have been developed to further converge the stabilized control state at higher speed.

FIG. 5A shows the case where the direct LMS algorithm is applied to a sound obtained by supplying white noise to silent portions of noise of snoring, and the application of the algorithm is temporarily stopped at portions of the sound that have low noise levels. FIG. 5B shows the case where the algorithm is applied to the entire sound. In FIGS. 5A and 5B, the black lines indicate the amplitude before the control, and the gray lines indicate the amplitude after the control. In FIG. 5A, the application of the algorithm is stopped when the number of update operations ranges from 28000 to 39000 and from 47000 to 62000, and exceeds 69000, as is indicated by the arrows. From comparison of FIGS. 5A and 5B, it can be understood that FIG. 5A, in which control is intermittently stopped, exhibits a superior control effect. In particular, the 4^{th} noise of snoring shows a significant difference therebetween in control effect. Namely, the difference in amplitude between before and after the control is more remarkable in FIG. 5A than in FIG. 5B.

### SECOND EXAMPLE

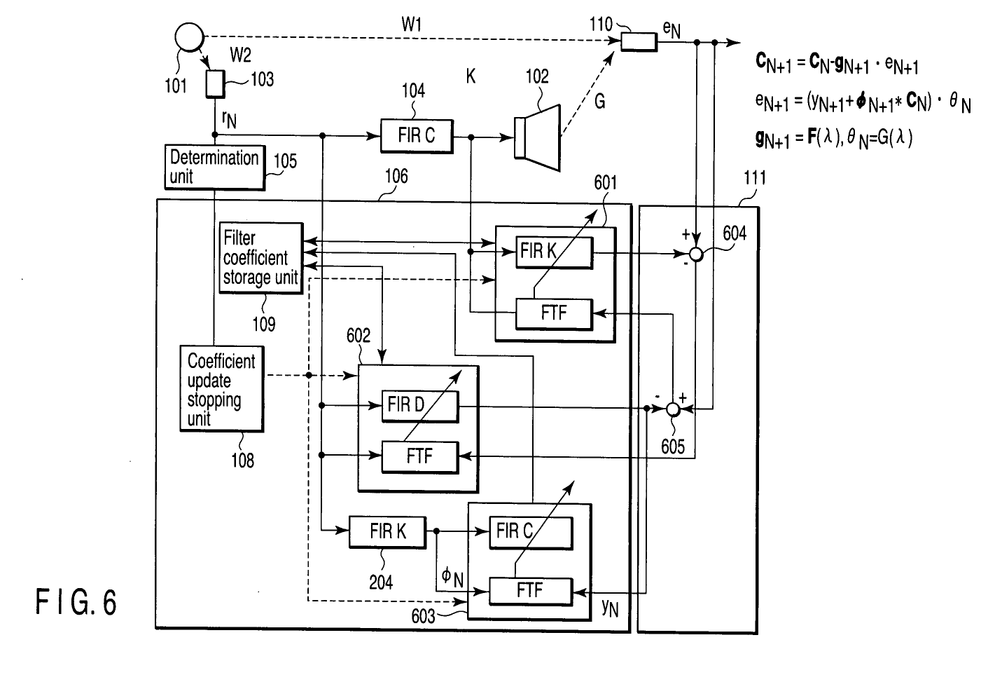

Referring to FIG. 6, a description will be given of a second example of the active noise-reduction control apparatus according to the first embodiment.

The second example differs from the first example of FIG. 2 in the adaptive filter unit and signal computation unit 111. Specifically, the second example employs adaptive filter units 601, 602 and 603 instead of the adaptive filter units 201, 202 and 203. The adaptive filter unit 601 includes a control filter K and FTF computation unit, the adaptive filter unit 602 includes a control filter D and FTF computation unit, and the adaptive filter unit 603 includes a control filter C and FTF computation unit. Further, signal computation unit 111 computes a signal necessary to update the coefficients the adaptive filter units 601 to 603 based on the outputs of the two adaptive filter units 601 and 602 and the output of the error microphone 110. In the second example, an FTF method is applied to adaptive-filter-coefficient update computation in order to suppress the sound pressure in the error microphone 110.

The FTF method is an adaptive algorithm that uses a high-speed transversal filter and belongs to a least square method. The amplitude convergence speed of the FTF method is higher than that of the above-described gradient-type LMS method, although the former requires a larger number of computations than the latter. Accordingly, in this method, if a reference signal falling outside a preset threshold value range is input and the control effect is degraded, it is effective to initialize the coefficient.

The FTF algorithm is disclosed in “Adaptive Signal Processing Algorithms” (written by Yoji Iikuni, published by Baifukan Publisher, Tokyo, July 2000, chuo-gaku, 547.1/I 11325274, pp. 172-175), and hence is not disclosed in detail. The FTF method, shown in FIG. 6, of computing an error signal from two signals input to the FTF computation units, and updating the filters is similar to the LMS method.

However, the FTF method significantly differs from the LMS method in that in the former, coefficient update computation is complex and uses constant λ that is not used in the direct LMS method, to control the convergence of coefficients. This will be described briefly using the following equations:
<C>_{N+1}=<C>_{N}−<g>_{N+1}·e_{N+1}
e_{N+1}=(y_{N+1}+<φ>_{N+1}*<C>_{N})·θN
<g>_{N+1}=<F(λ)>, θN=G(λ) (Eq. 4)
where <A>*<B> indicates the scalar product of vectors A and B.

Concerning update of the coefficient of the filter C in FIG. 6, update computation is performed in the following manner. <g>_{N} and e_{N} (scalar value) correspond to <s>_{N} and e2_{N} in the LMS method, respectively. In the LMS method, update of the filter C is directly performed using those values. In contrast, in the FTF method, e is computed from an input signal y_{N} (called a target value in the FTF method) input to the adaptive filter unit 603 from the right hand, and an input signal φ_{N} input thereto from the left hand. Further, <g>_{N} is computed using a more complex virtual error computation expression. During the computation of e, constant λ is used.

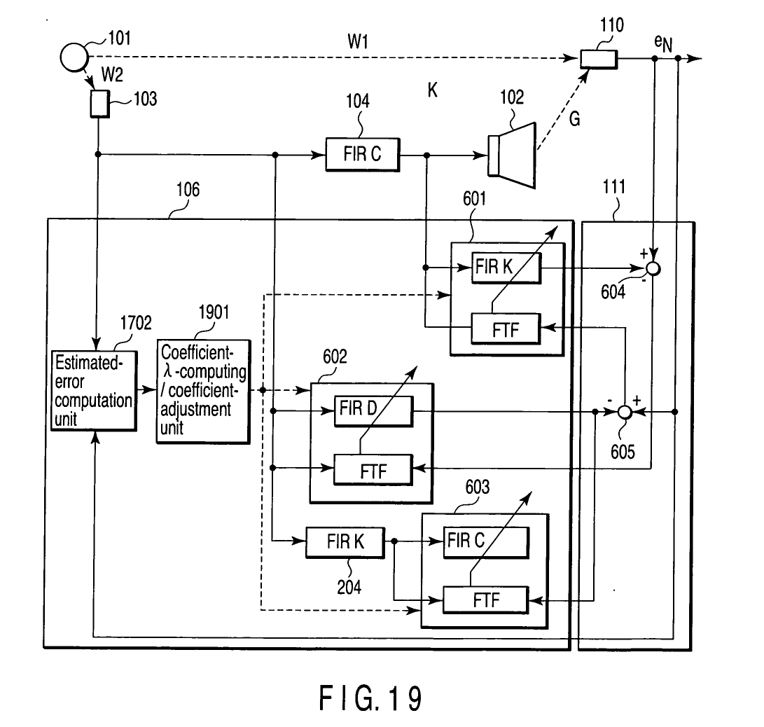

In the above FTF method applied to a steady sound, λ is input as a constant (fixed value). However, λ is varied during control of an unsteady sound, which will be described later with reference to FIG. 19.

### Second Embodiment

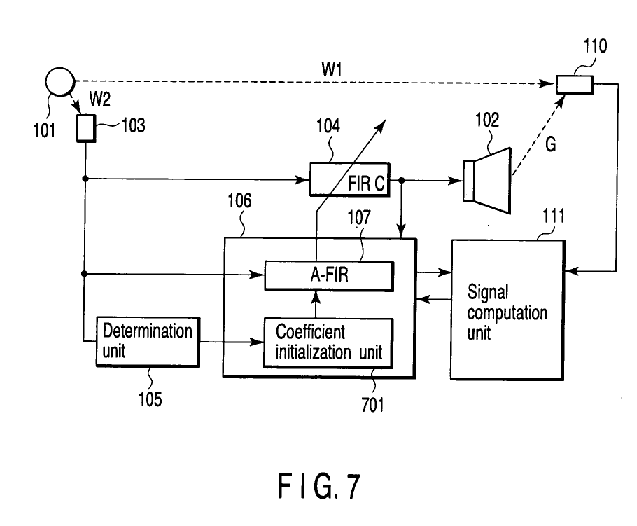

Referring to FIG. 7, an active noise-reduction control apparatus according to a second embodiment of the invention will now be described.

The active noise-reduction control apparatus of the second embodiment differs from that of the first embodiment only in the internal structure of the filter coefficient update unit 106. The filter coefficient update unit 106 of the second embodiment comprises an adaptive filter unit 107 and coefficient initialization unit 701.

The coefficient initialization unit 701 initializes the coefficient of the digital filter arithmetic unit 104 when a change in the level of the reference signal output from the reference signal generator 103 falls outside the threshold value range. Namely, in the case of, for example, such a general adaptive filter as shown in FIG. 1B, the coefficient C of the control filter is once initialized to zero. See, for example, the above-mentioned equation (Eq. 1).

As described above, the second embodiment is characterized in that the sound pressure in the error microphone 110 is suppressed without identifying the error route (spatial transmission function) between the error microphone 110 and to-be-reduced noise 101, and in that outside the threshold-value range set in the determination unit 105, the filter coefficient of the filter coefficient update unit 106 is initialized, thereby realizing suppression of error-microphone sound pressure even if the to-be-reduced noise 101 is an unsteady sound of a greatly varying level, intermittent sound emitted from a sound source that intermittently stops, or sound emitted from a moving sound source.

### FIRST EXAMPLE

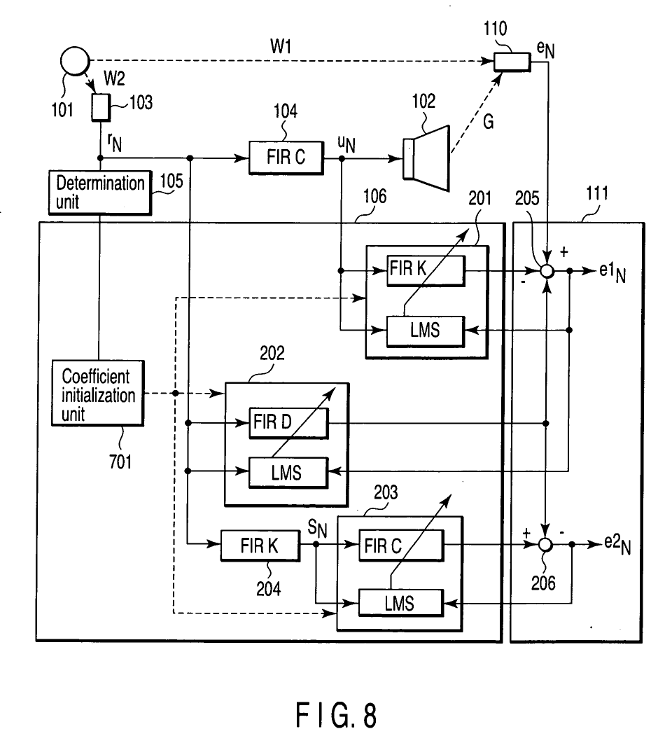

Referring to FIG. 8, a first example of the active noise-reduction control apparatus of the second embodiment will be described.

This example is acquired by providing the first example of the first embodiment with the coefficient initialization unit 701 instead of the coefficient update stopping unit 108, and further excluding therefrom the filter coefficient storage unit 109.

The coefficient initialization unit 701 initializes all control coefficients when the sound pressure of the noise source is significantly varied to a value falling outside the threshold-value range set by the determination unit 105. Initialization is the process of making all the coefficients zero. At this time, at least the control filter C must be initialized. Further, the remaining control filters K and D may be initialized as well as the control filter C.

When the adaptive filter units 201, 202 and 203 perform coefficient update computation, even if they set all control filter coefficients to zero to perform update control from the beginning, they can suppress the sound pressure in the error microphone 110 and maintain the suppressed state without degrading the noise-reduction effect of the error microphone 110, by utilizing the LMS method instead of the conventional Filtered-X method.

### SECOND EXAMPLE

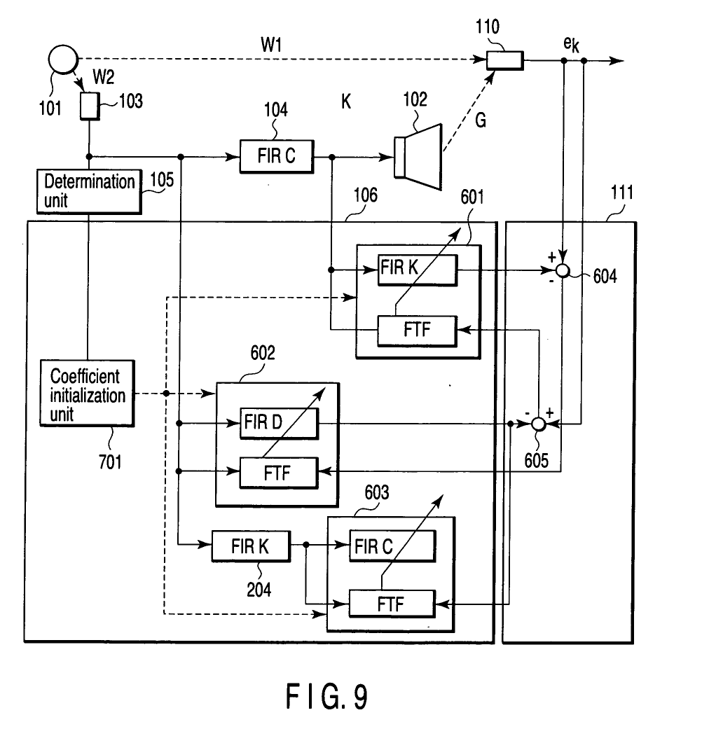

Referring to FIG. 9, a description will be given of a second example of the active noise-reduction control apparatus according to the second embodiment.

The second example differs from the first example of FIG. 8 in the adaptive filter unit and signal computation unit 111. Specifically, the second example employs adaptive filter units 601, 602 and 603 instead of the adaptive filter units 201, 202 and 203. The adaptive filter unit 601 includes a control filter K and FTF computation unit, the adaptive filter unit 602 includes a control filter D and FTF computation unit, and the adaptive filter unit 603 includes a control filter C and FTF computation unit. Further, signal computation unit 111 computes a signal necessary to update the coefficients the adaptive filter units 601 to 603 based on the outputs of the two adaptive filter units 601 and 602 and the output of the error microphone 110. In the second example, in which an FTF method is utilized, even if all control filter coefficients are once set to zero, the original state can be quickly recovered.

In the second example, the FTF method can be utilized, and even if the noise-reduction effect of the error microphone 110 is degraded, the control results do not diverge, thereby realizing suppression of the sound pressure in the error microphone 110 and maintaining the suppressed state. Since the FTF method can realize quicker amplitude convergence than the direct LMS method, the method of the second example, in which all control coefficients are once initialized, can be used as the most effective means when the noise reduction effect is degraded by the input of an error signal that falls outside the threshold-value range.

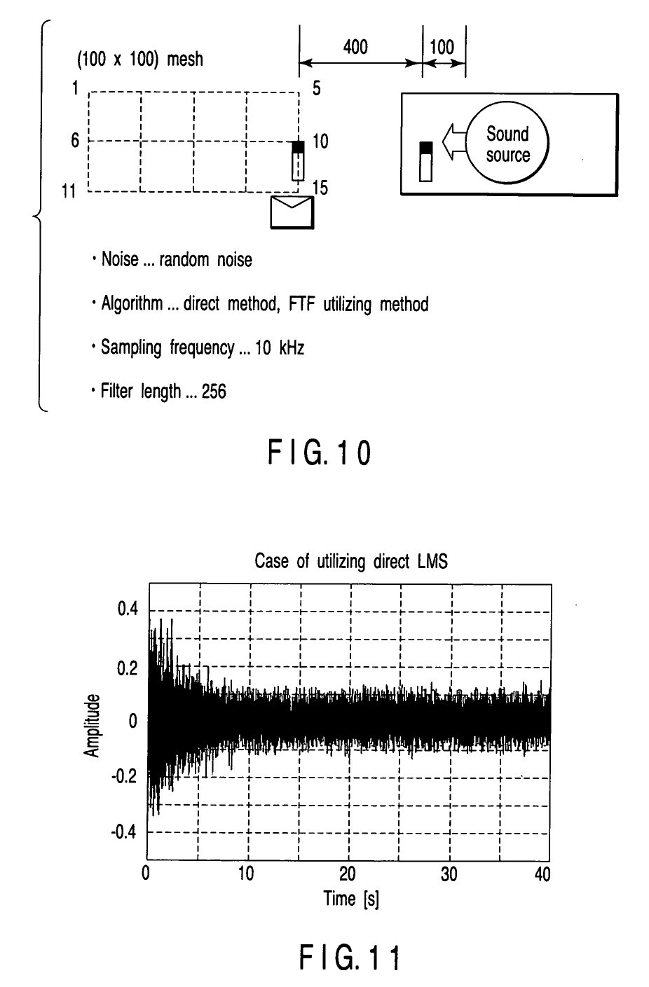

Referring to FIGS. 10 to 14, a description will be given of the experiments actually conducted. The validity of the direct FTF method and direct LMS method applied to random noise of 5 kHz or less was estimated by the experimental system shown in FIG. 10, using a sampling frequency of 10 kHz and a cutoff frequency (LPF) of 4 kHz.

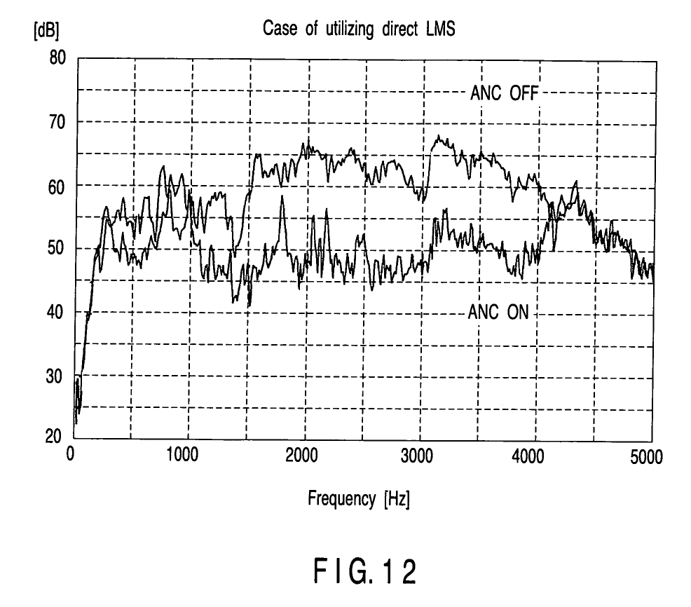

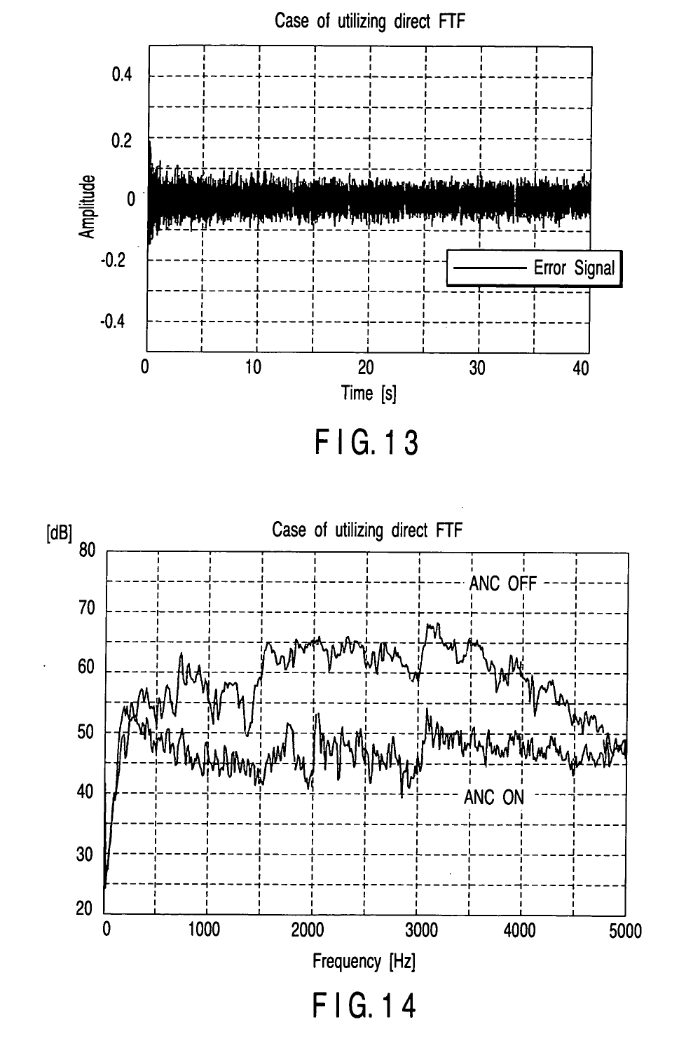

The validity of the direct LMS method will be described with reference to FIGS. 11 and 12, and that of the direct FTF method will be described with reference to FIGS. 13 and 14. FIGS. 11 and 13 each show the time-series waveform of the sound output from the error microphone 110. In these figures, the horizontal axis indicates the time. FIGS. 12 and 14 each show the control effect of the error microphone 110. In these figures, the horizontal axis indicates the frequency. More specifically, each of FIGS. 12 and 14 shows the noise reduction effect acquired when the active noise-reduction control apparatus is in the ON state (ANC on), and that acquired when the apparatus is in the OFF state (ANC off). It can be understood from FIGS. 11 to 14 that in the FTF method, the noise amplitude is converged within one second, and a greater noise reduction is detected in a wider band than in the LMS method.

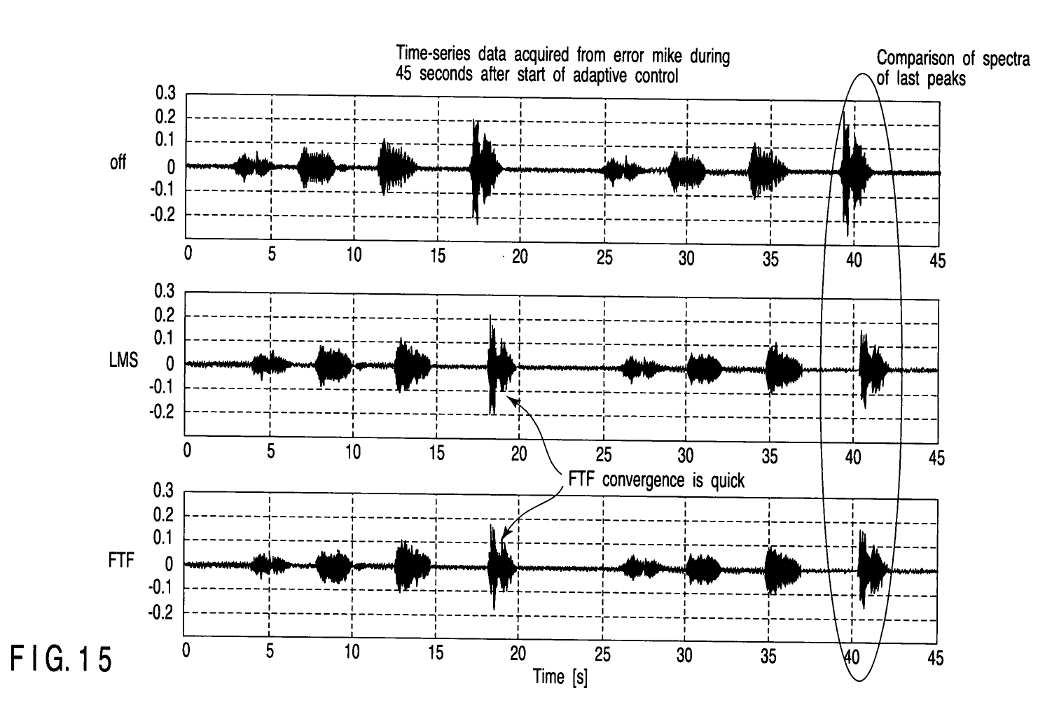

Referring to FIG. 15, unsteady noise of snoring will be described. FIG. 15 shows results acquired when the active noise-reduction control apparatus is in the OFF state, the LMS method is utilized, and the FTF method is utilized. The results are time-series data acquired from the error microphone 110 within 45 seconds after the start of adaptive control, with the sampling frequency set to 10 kHz and the cutoff frequency (LPF) set to 3.5 kHz. From FIG. 15, it can be understood that the direct FTF method realizes quicker amplitude convergence.

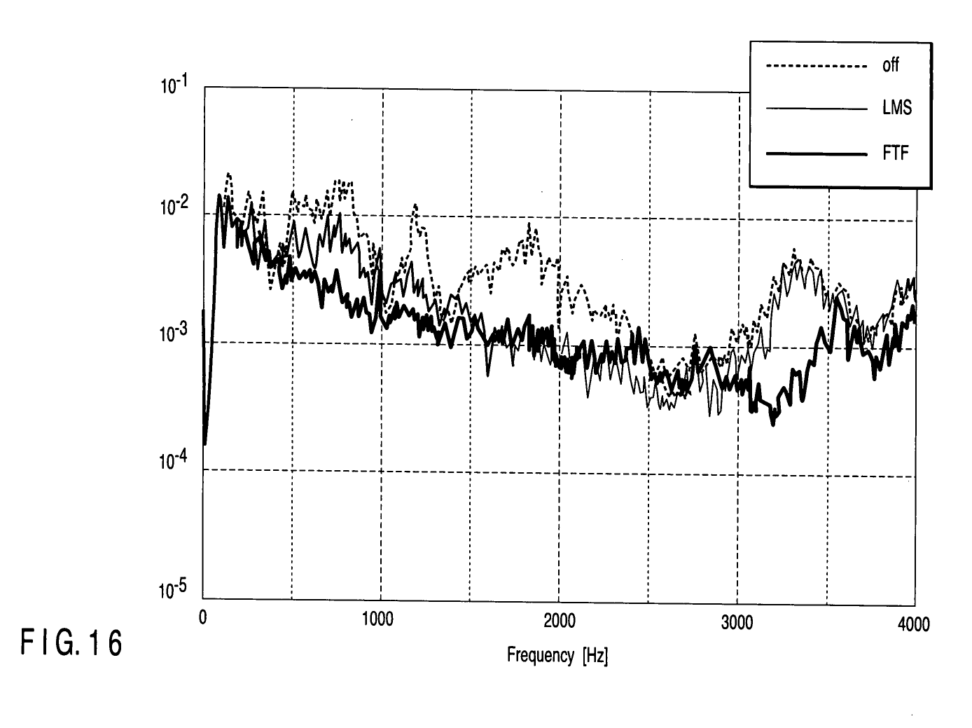

Referring to FIG. 16, the control effect of the error microphone 110 will be described. FIG. 16 shows the control effect of the error microphone 110, the horizontal axis indicating the frequency. It can be understood from FIG. 16 that the FTF method is effective over substantially the entire frequency band, and exhibits a conspicuous advantage at, in particular, a high-frequency band (3 to 4 kHz), compared to the LMS method.

### Third Embodiment

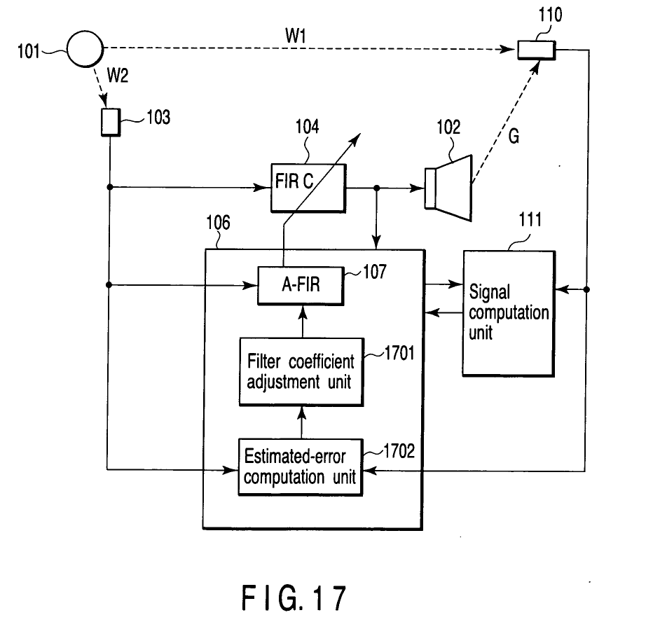

Referring to FIG. 17, an active noise-reduction control apparatus according to a third embodiment of the invention will be described.

The active noise-reduction control apparatus of the third embodiment differs from that of the first embodiment only in the internal structure of the filter coefficient update unit 106. The filter coefficient update unit 106 of the third embodiment comprises a filter coefficient adjustment unit 1701 and estimated-error computation unit 1702.

The estimated-error computation unit 1702 computes an estimated error EE based on a reference signal from the reference signal generator 103 and an error signal from the error microphone 110. The estimated error EE is expressed by
EE=10 log₁₀(Σe²/Σd²) (Eq. 5)
where e is an error microphone signal, and d is a reference microphone signal.

The filter coefficient adjustment unit 1701 adjusts the prestored coefficient of the adaptive filter unit 107 based on the estimated error EE. As a result, control can be achieved, while varying the coefficient in accordance with the level of the error microphone signal that varies with time.

In the third embodiment, even noise that greatly varies in level, or a sound emitted from a moving sound source can be controlled reliably without amplitude divergence. In the reference-signal-level determination method employed in the first and second embodiments, it is necessary to accurately set a threshold value. In contrast, in the third embodiment, no determination unit 105 is necessary, and hence no such setting is necessary, either. In this point, more stable control can be realized.

### FIRST EXAMPLE

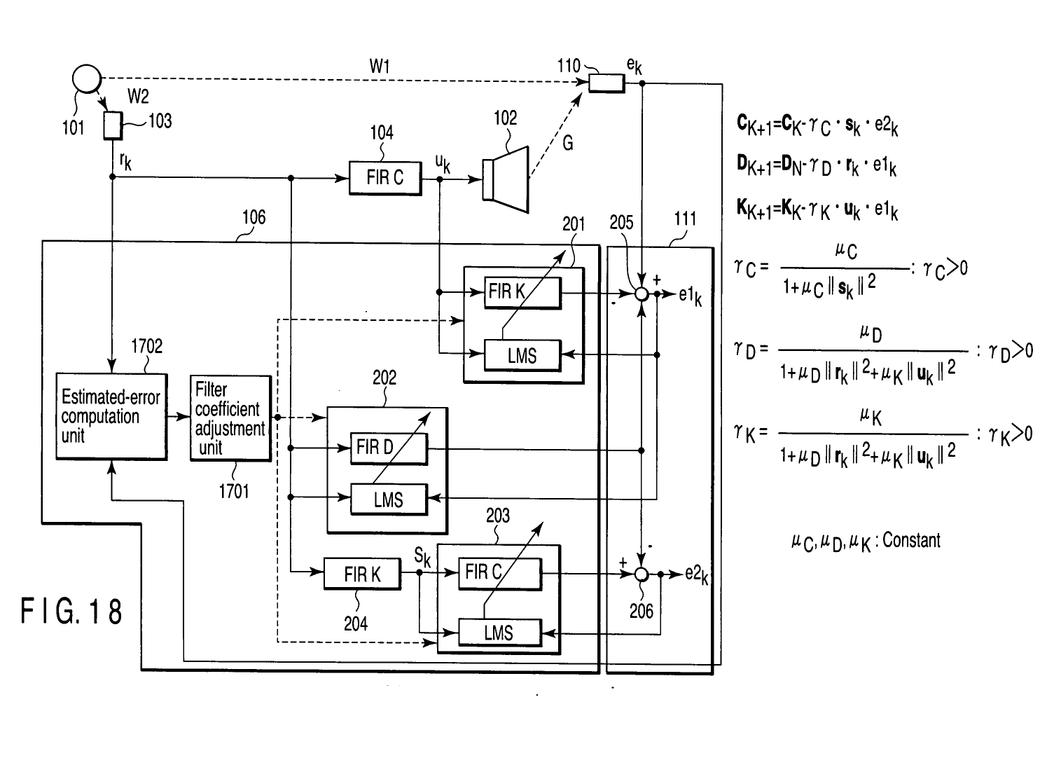

Referring to FIG. 18, a first example of the active noise-reduction control apparatus of the third embodiment will be described.

This example is acquired by providing the first example of the first embodiment with the estimated-error computation unit 1702 and filter coefficient adjustment unit 1701 instead of the coefficient update stopping unit 108, and further excluding therefrom the filter coefficient storage unit 109.

The estimated-error computation unit 1702 computes an estimated error EE based on a reference signal and an error signal, and the filter coefficient adjustment unit 1701 varies, using the estimated error EE, μ_{C}, μ_{D} and μ_{K} (these values are constants in the conventional direct LMS method). As a result, stable control can also be realized, without control divergence, concerning noise of a greatly varying level or a sound emitted from a moving sound source.

### SECOND EXAMPLE

Referring to FIG. 19, a description will be given of a second example of the active noise-reduction control apparatus according to the third embodiment.

The second example differs from the first example of FIG. 18 in the adaptive filter unit, signal computation unit 111 and filter coefficient adjustment unit 1701. Specifically, the second example employs a coefficient-λ-computing/coefficient-adjustment unit 1901 instead of the filter coefficient adjustment unit 1701.

The estimated-error computation unit 1702 computes an estimated error EE from a reference signal and an error signal, and the coefficient-λ-computing/coefficient-adjustment unit 1901 varies λ based on the estimated error EE (λ is a constant as a forgetting coefficient, e.g., 0.999, in the conventional direct FTF method).

For instance, the estimated error EE and λ have the following relationship:
EEE=κ·EEE+(1−κ)·EE, κ=0.999

This equation means that EEE at the left-hand side, i.e., a new value, is acquired by updating EEE and EE at the right-hand side, i.e., present values.
λ=1-10(−3.7+EEE/15)

This equation of λ is just an example.

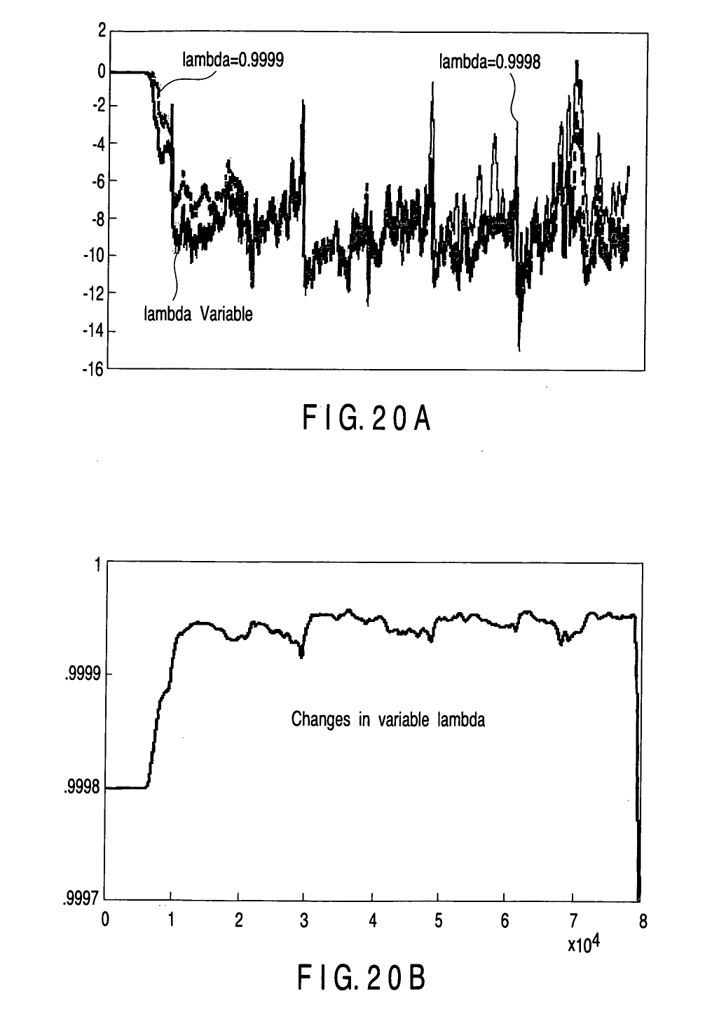

Referring then to FIGS. 20A and 20B, a description will be given of the control effect acquired when coefficient λ is updated using the equation. In FIGS. 20A and 20B, the horizontal axis indicates the number of times of control sampling (i.e., the elapsed time). Further, the vertical axis in FIG. 20A indicates the noise reduction amount (dB) of the error microphone before and after control. The vertical axis of FIG. 20B indicates variable λ. It can be understood from FIGS. 20A and 20B that the noise is more reduced without divergence in the case of using variable λ, than in the case of using fixed λ.

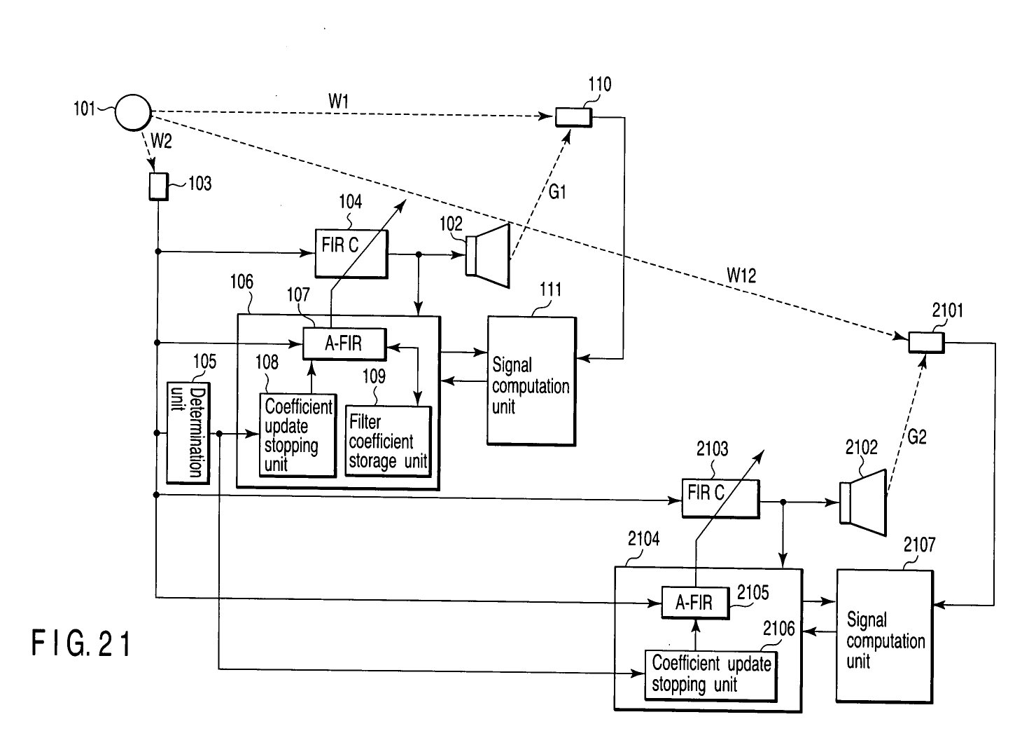

Referring to FIG. 21, an active noise-reduction control apparatus according to a fourth embodiment of the invention will be described.

The active noise-reduction control apparatus of the fourth embodiment uses two error microphones 110 and 2101 to simultaneously suppress the sound pressure of the two microphones.

The active noise-reduction control apparatus of the fourth embodiment comprises an error microphone 2101, control sound source unit 2102, digital filter arithmetic unit 2103, filter coefficient update unit 2104 and signal computation unit 2107. The filter coefficient update unit 2104 includes an adaptive filter unit 2105 and coefficient update stopping unit 2106. The new elements shown in FIG. 21 have the same functions as the elements having similar names in FIG. 1.

In the fourth embodiment, sound pressure in the error microphones 110 and 2101 are simultaneously suppressed without identifying the error routes (spatial transmission functions) between the error microphone 110 and the control-sound source unit 102 and between the error microphone 2101 and the control sound source unit 2102. Concerning the noise falling outside the threshold-value range set in the determination unit 105, noise pressure in the error microphones 110 and 2101 is simultaneously suppressed by stopping the coefficient changing operations of the filter coefficient updating units 106 and 2104, even if the to-be-reduced noise 101 is an unsteady sound of a greatly varying level, intermittent sound (including silent portions) emitted from a sound source that intermittently stops, or sound emitted from a moving sound source.

When the two error microphones are positioned close to the ears and noise reduction at the ears is aimed at, and when the distances between the error microphones and the speakers are long, it is difficult to simultaneously reduce the noise in the error microphones unless a control algorithm made in consideration of the influence of crosstalk and the acoustic transmission function of the crosstalk is utilized. However, the fourth embodiment can simultaneously reduce the noise in the error microphones simply by using the single reference microphone, i.e., the reference signal generator 103, for the error microphones in common.

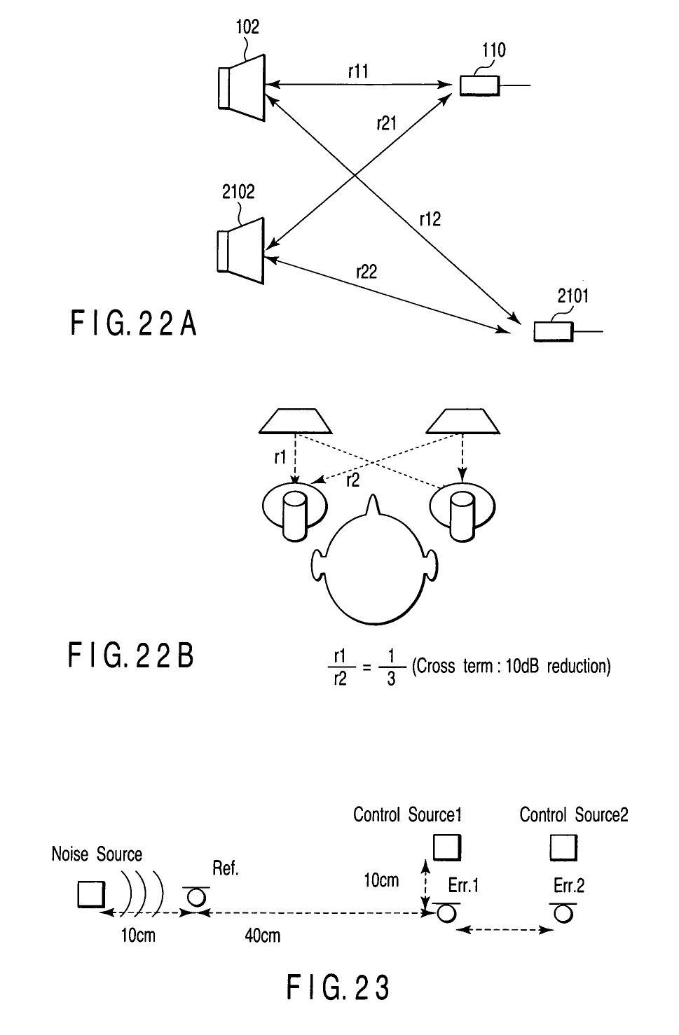

In particular, as shown in FIGS. 22A and 22B, assume that the distance r21 between the control sound source unit 2102 and the error microphone 110 is substantially three times or more the distance r11 between the adjacent control-sound source unit 102 and the error microphone 110, and that the distance r12 between the control-sound source unit 102 and the error microphone 2101 is substantially three times or more the distance r22 between the adjacent control sound source unit 2102 and the error microphone 2101. In this case, since the distance ratio is 3, noise of crosstalk is reduced by about 10 dB (=10 log₁₀ 3²) in light of the fact that the sound pressure attenuates reversely proportional to the distance. Accordingly, an algorithm that does not consider noise of crosstalk is applicable to this case.

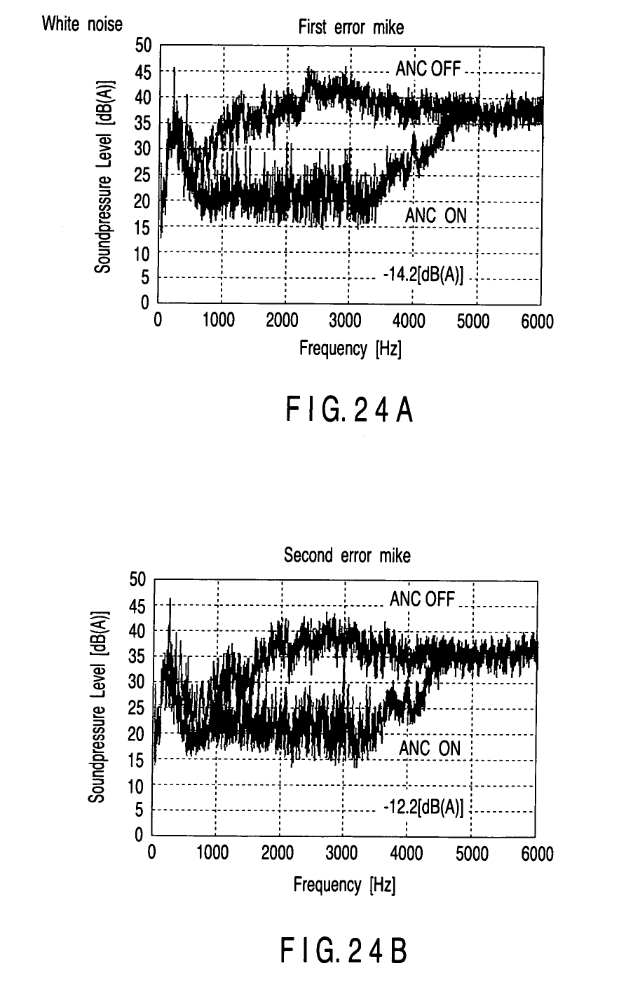

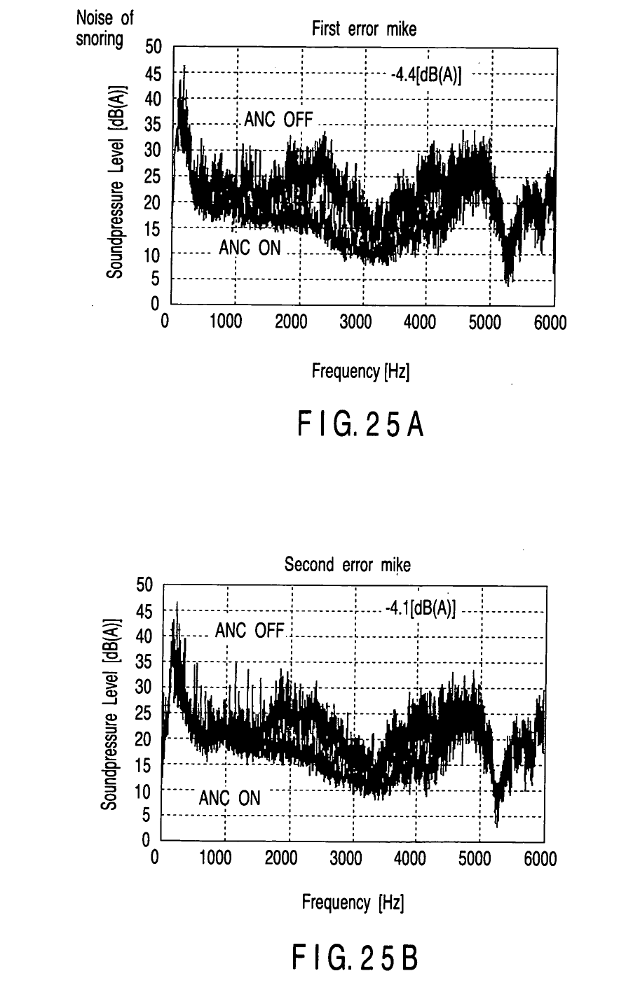

Referring to FIGS. 24A and 24B, a description will be given of the results acquired when the experimental system shown in FIG. 23 emits random noise (white noise). Further, the results acquired when the experimental system shown in FIG. 23 emits noise of snoring will be described referring to FIGS. 25A and 25B.

From FIGS. 24A to 25B, it is evident that the noise is sufficiently reduced from the low-frequency portion to the high-frequency portion thereof when the active noise-reduction control apparatus of the fourth embodiment is in the ON state (ANC on). The values in the figures indicate the amounts of reduction of noise (integral values) in the range of 200 Hz to 4 kHz. Noise of snoring is lower by about 10 dB than the random noise. This is because a great reduction of noise occurs at and around 200 Hz. Namely, in the noise of snoring, the reduction at and around 200 Hz significantly contributes to the integral value, whereas in the random noise, the reduction at and around 200 Hz does not significantly contribute to the integral value.

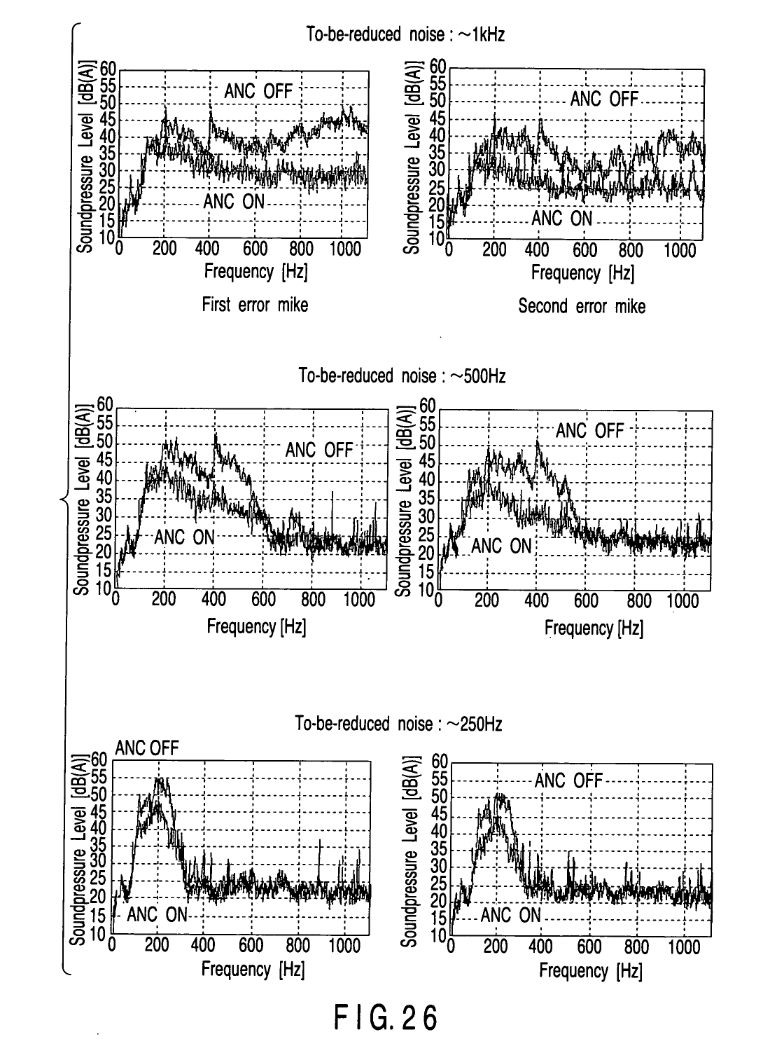

Referring to FIG. 26, a description will be given of the results of control performed on the noise of snoring under the same conditions as in FIGS. 25A and 25B but in smaller bands of frequency. It can be understood from FIG. 26 that in any frequency band, the two error microphones sufficiently reduce to-be-reduced noise, namely, the quasi-2-channel algorithm employed in the fourth embodiment is effective.

As described above, in the embodiments, a change in to-be-reduced noise is determined from the level of a reference signal (absolute voltage) and a change in the level (relative voltage). In a certain period of time, control is performed with the coefficient of the control filter C fixed, and when the effect of control is degraded, control is performed with the control filter C initialized, i.e., returned to the state before the control. As a result, variation in noise level and movement of the noise source can be followed.

In addition, in the embodiments, an estimated error value related to the effect of control is computed using an error signal and reference signal, and is used to perform fine adjustment of the filter coefficients. This enables more stable control and quicker noise-level-converging control that can follow even unsteady sound of a greatly varying level or sound emitted from a sound source moving at high speed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An active noise-reduction control apparatus comprising:
a reference signal generator which generates a reference signal based on noise emitted from a sound source;
a detector which detects a level of the reference signal and a change in the level;
a comparison unit configured to compare the change with a threshold-value range and produce a compared result;
an adaptive filter configured to filter the reference signal, the adaptive filter having a variable filter coefficient;
an updating unit configured to update the filter coefficient according to the change of the level of the reference signal for obtaining an updated filter coefficient;
a stop unit configured to stop updating of the filter coefficient in response to the compared result when the change falls outside the threshold-value range;
a storage unit configured to store the updated filter coefficient each time the filter coefficient is updated;
a control signal generator which generates a control signal using the stored filter coefficient;
a control-sound source unit configured to generate a control sound based on the control signal;
an error microphone which detects synthesis sound pressure of the control sound and the noise to produce an error signal; and
a setting unit configured to set the stored filter coefficient to a more accurate coefficient than the stored filter coefficient based on the error signal, and a signal acquired by filtering the control signal through the adaptive filter.

2. The apparatus according to claim 1, wherein the setting unit is configured to set the stored filter coefficient using a least mean square (LMS) algorithm.

3. The apparatus according to claim 1, wherein the setting unit is configured to set the stored filter coefficient using a fast transversal filter (FTF) algorithm.

4. An active noise-reduction control apparatus comprising:
a reference signal generator which generates a reference signal based on noise emitted from a sound source;
a detector which detects a level of the reference signal and a change in the level;
a comparison unit configured to compare the change with a threshold-value range and produce a compared result;
an adaptive filter configured to filter the reference signal, the adaptive filter having a variable filter coefficient;
an initialization unit configured to initialize the filter coefficient in response to the compared result when the change falls outside the threshold-value range;
a control signal generator which generates a control signal using the filter coefficient;
a control-sound source unit configured to generate a control sound based on the control signal;
an error microphone which detects synthesis sound pressure of the control sound and the noise to produce an error signal; and
a setting unit configured to set the filter coefficient to a more accurate coefficient than the filter coefficient based on the error signal, and a signal acquired by filtering the control signal through the adaptive filter.

5. The apparatus according to claim 4, wherein the setting unit is configured to set the filter coefficient using a least mean square (LMS) algorithm.

6. The apparatus according to claim 4, wherein the setting unit is configured to set the filter coefficient using a fast transversal filter (FTF) algorithm.

7. An active noise-reduction control apparatus comprising:
a reference signal generator which generates a reference signal based on noise emitted from a sound source;
a control-sound source unit configured to generate a control sound used for controlling reduction of the noise;
an error microphone which detects synthesis sound pressure of the control sound and the noise to produce an error signal;
a computation unit configured to compute an estimated error based on the reference signal and the error signal;
an adaptive filter configured to filter the reference signal, the adaptive filter having a variable filter coefficient;
an adjustment unit configured to adjust the filter coefficient based on the estimated error;
a control signal generator which generates a control signal using the filter coefficient; and
a setting unit configured to set the filter coefficient to a more accurate coefficient than the filter coefficient based on the error signal, and a signal acquired by filtering the control signal through the adaptive filter,
the control-sound source unit generating the control sound based on the control signal.

8. The apparatus according to claim 7, wherein the setting unit is configured to set the filter coefficient using a least mean square (LMS) algorithm.

9. The apparatus according to claim 7, wherein the setting unit is configured to set the filter coefficient using a fast transversal filter (FTF) algorithm.

10. An active noise-reduction control apparatus comprising:
a reference signal generator which generates a reference signal based on noise emitted from a sound source;
a detector which detects a level of the reference signal and a change in the level;
a comparison unit configured to compare the change with a threshold-value range and produce a compared result;
a first adaptive filter configured to filter the reference signal, the first adaptive filter having a first variable filter coefficient;
a first updating unit configured to update the first variable filter coefficient according to the change of the level of the reference signal for obtaining an updated first variable filter coefficient;
a first stop unit configured to stop updating of the first variable filter coefficient in response to the compared result when the change falls outside the threshold-value range;
a first control signal generator which generates a first control signal using the first variable filter coefficient;
a first control-sound source unit configured to generate a first control sound based on the first control signal;
a first error microphone which detects first synthesis sound pressure of the first control sound and the noise to produce a first error signal;
a first setting unit configured to set the first variable filter coefficient to a more accurate coefficient than the first variable filter coefficient based on the first error signal, and a signal acquired by filtering the first control signal through the first adaptive filter;
a second adaptive filter configured to filter the reference signal, the second adaptive filter having a second variable filter coefficient;
a second updating unit configured to update the second variable filter coefficient according to the change of the level of the reference signal for obtaining an updated second variable filter coefficient;
a second stop unit configured to stop updating of the second variable filter coefficient in response to the compared result when the change falls outside the threshold-value range;
a second control signal generator which generates a second control signal using the second variable filter coefficient;
a second control-sound source unit configured to generate a second control sound based on the second control signal;
a second error microphone which detects second synthesis sound pressure of the second control sound and the noise to produce a second error signal;
a second setting unit configured to set the second variable filter coefficient to a more accurate coefficient than the second variable filter coefficient based on the second error signal, and a signal acquired by filtering the second control signal through the second adaptive filter.

11. The apparatus according to claim 10, wherein the first setting unit and the second setting unit are configured to set the filter coefficient a least mean square (LMS) algorithm.

12. The apparatus according to claim 10, wherein the first setting unit and the second setting unit are configured to set the filter coefficient using a fast transversal filter (FTF) algorithm.

13. An active noise-reduction control method comprising:
generating a reference signal based on noise emitted from a sound source;
detecting a level of the reference signal and a change in the level;
comparing the change with a threshold-value range and producing a compared result;
preparing an adaptive filter configured to filter the reference signal, the adaptive filter having a variable filter coefficient;
updating the filter coefficient according to the change of the level of the reference signal for obtaining an updated filter coefficient;
stopping updating of the filter coefficient in response to the compared result when the change falls outside the certain threshold-value range;
preparing a storage unit configured to store the updated filter coefficient each time the filter coefficient is updated;
generating a control signal using the stored filter coefficient;
generating a control sound based on the control signal;
detecting synthesis sound pressure of the control sound and the noise to produce an error signal; and
setting the stored filter coefficient to a more accurate coefficient than the stored filter coefficient based on the error signal, and a signal acquired by filtering the control signal through the adaptive filter.

14. An active noise-reduction control method comprising:
generating a reference signal based on noise emitted from a sound source;
detecting a level of the reference signal and a change in the level;
comparing the change with a threshold-value range and producing a compared result;
preparing an adaptive filter configured to filter the reference signal, the adaptive filter having a variable filter coefficient;
initializing the filter coefficient in response to the compared result when the change falls outside the threshold-value range;
generating a control signal using the filter coefficient;
generating a control sound based on the control signal;
detecting synthesis sound pressure of the control sound and the noise to produce an error signal; and
setting the filter coefficient to a more accurate coefficient than the filter coefficient based on the error signal, and a signal acquired by filtering the control signal through the adaptive filter.

15. An active noise-reduction control method comprising:
generating a reference signal based on noise emitted from a sound source;
generating a control sound used for controlling reduction of the noise;
detecting synthesis sound pressure of the control sound and the noise and producing an error signal;
computing an estimated error based on the reference signal and the error signal;
preparing an adaptive filter configured to filter the reference signal, the adaptive filter having a variable filter coefficient;
adjusting the filter coefficient based on the estimated error;
generating a control signal using the filter coefficient; and
setting the filter coefficient to a more accurate coefficient than the filter coefficient based on the error signal, and a signal acquired by filtering the control signal through the adaptive filter,
generating the control sound based on the control signal.

16. An active noise-reduction control method comprising:
generating a reference signal based on the noise emitted from a sound source;
detecting a level of the reference signal and a change in the level;
comparing the change with a threshold-value range and producing a compared result;
preparing a first adaptive filter configured to filter the reference signal, the first adaptive filter having a first variable filter coefficient;
updating the first variable filter coefficient according to the change of the level of the reference signal for obtaining an updated first variable filter coefficient;
stopping updating of the first variable filter coefficient in response to the compared result when the change falls outside the threshold-value range;
generating a first control signal using the first variable filter coefficient;
generating a first control sound based on the first control signal;
detecting first synthesis sound pressure of the first control sound and the noise and producing a first error signal;
setting the first variable filter coefficient to a more accurate coefficient than the first variable filter coefficient based on the first error signal, and a signal acquired by filtering the first control signal through the first adaptive filter;
preparing a second adaptive filter configured to filter the reference signal, the second adaptive filter having a second variable filter coefficient;
updating the second variable filter coefficient according to the change of the level of the reference signal for obtaining an updated second variable filter coefficient;
stopping updating of the second variable filter coefficient in response to the compared result when the change falls outside the threshold-value range;
generating a second control signal using the second variable filter coefficient;
generating a second control sound based on the second control signal;
detecting second synthesis sound pressure of the second control sound and the noise to produce a second error signal;
setting the second variable filter coefficient to a more accurate coefficient than the second variable filter coefficient based on the second error signal, and a signal acquired by filtering the second control signal through the second adaptive filter.
